SEQUENCE LISTING (3 sequences)

SEQ ID NO: 1 - protein, 9 aa, Artificial Sequence
  Artificially synthesized GluR2-3Y peptide
YKEGYNVYG

SEQ ID NO: 2 - protein, 11 aa, Artificial Sequence
  Artificially synthesized Tat peptide
YGRKKRRQRRR

SEQ ID NO: 3 - protein, 20 aa, Artificial Sequence
  Artificially synthesized Tat-GluR2-3Y peptide
YGRKKRRQRRRYKEGYNVYG

(19) United States
(12) Patent Application Publication
(10) Pub. No.: US 2022/0347266 A1
(43) Pub. Date: Nov. 3, 2022

(54) METHOD AND MEDICINE FOR TREATING DEPRESSIVE DISORDERS

(71) Applicant: QINGDAO PRIMEDICINE PHARMACEUTICAL CO., LTD., Qingdao (CN)

(72) Inventors: Yutian Wang, Vancouver (CA); Yang Li, Shanghai (CN); Fei Guo, Shanghai (CN)

(21) Appl. No.: 17/436,900

(22) PCT Filed: Nov. 21, 2019
(86) PCT No.: PCT/CN2019/119855

§ 371 Date: Jun. 21, 2022

(30) Foreign Application Priority Data
Nov. 21, 2018 (CN) 201811390067.5

(51) Int. Cl. A61K 38/17; A61P 25/24

(52) U.S. Cl. CPC A61K 38/1787; A61P 25/24

(57) ABSTRACT

Disclosed are a method and medicine for treating depressive disorders. In particular, disclosed is a use of a GluR2-CT polypeptide for treating depressive disorders, specifically, major depressive disorder.

### TECHNICAL FIELD

The invention relates to the field of biomedicine. In particular, the invention discloses a novel method and medicament for treating depressive disorder. More particularly, the present invention relates to a novel use of GluR2-CT polypeptide in the treatment of depressive disorder, specifically major depressive disorder.

### BACKGROUND ART

Depressive disorder is a global and clinically common mental health disorder, which is especially likely to occur in people experiencing stroke, neurasthenia, organ transplantation, diabetes, Parkinson's disease, neurosis, and postpartum and elderly people. If the condition is serious and recurring in a patient, suicidality will appear, which will affect the patient's daily life. Based on the statistics of the World Health Organization, depressive disorder has presently become the fourth prevalent disease in the world, and may become the second disease after ischemic heart disease making patients incapable of self-care by 2020. Depressive disorder is very common in clinical treatment, which is mainly caused by severe social and emotional disorders of patients.

Depressive disorder may be divided into mild/moderate depressive disorder and major depressive disorder. Clinical manifestations for mild/moderate depressive disorder include: having a depressed mood or other symptoms (such as changes in appetite and sleep patterns, unhappiness, pessimism, feeling of inferiority, etc.) for more than two years without reaching the level of major depressive disorder; and living in an unnormal state daily for two months or more. Clinical manifestations for major depressive disorders include: weight gain or weight loss when not dieting; change in appetite; sleeping too much or too little; feeling restless; lack of energy; feeling worthless, ashamed, and guilty; difficulty in concentrating and making decisions; and thoughts of death or suicide. If such symptoms occur in most of the day or last for two weeks or more in a person, then he is more likely to suffer from major depressive disorder.

Although patients with depressive disorder are in remission by taking conventional antidepressants, slow onset of action thereof may hamper their wide clinical applications. It often takes 3-4 weeks for conventional antidepressants to exert corresponding therapeutic effects, which not only increases the treatment period for patients leading to a decreased patient compliance, but also greatly lowers the quality of life and living state of patients. More seriously, for patients with major depressive disorder, the risk of suicide will be significantly increased due to the lack of timely and effective treatment, which will bring serious consequences to patients themselves and their families. Therefore, to exert the therapeutic effect of antidepressants in the shortest time to reduce the occurrence of associated risk has been an obstacle for the further development of antidepressants. Although the problem of slow onset of action has been solved by medications in development represented by esketamine, partly reducing the treatment period for patients with major depressive disorder, further clinical application of these medications are hampered by strong hallucinatory effect. In addition, common side effects of conventional antidepressants include dry mouth, constipation, sleepiness, weight gain, and hepatotoxicity, etc., which further hampers the use thereof. However, with the development of society, the increasing of stress in life as well as the increasing number of people with depressive disorder bring an urgent need for a safe medicament having a rapid action to meet the market need.

### BRIEF DESCRIPTION OF THE DRAWINGS

FIG. 1 shows the evaluation results of forced swim test. The left panel shows the forced swim test 1 hour after administration, and the right panel shows the forced swim test 24 hours after administration.

### SUMMARY OF THE INVENTION

The C-terminal polypeptide of GluR2 subunit (GluR2-CT polypeptide) of receptor AMPAR has been previously proved to be associated with anxiety (see EP1,687,427B1), for example, GluR2-CT polypeptide can block mood-induced stress anxiety in the elevated cross maze experiment on behavior model of animals with anxiety disorder, thereby being used for treating anxiety disorder. However, anxiety disorder clearly differs from depressive disorder. Anxiety is an expecting reaction caused by an inevitable and upcoming stress event known in advance, which mainly manifests as mental symptoms such as fear, worry, and tension, accompanied by autonomic nervous dysfunctions such as palpitation, sweating, and cold hands and feet, with the core symptom being worry. Depressive disorder, however, is a mental disorder mainly manifesting as depressed mood, and slowness of thought and behavior, which can be accompanied by physical symptoms such as less sleep and weight loss. Uncontrollable stress is one important reason that causes depressive disorder, that is to say, depressive behavior is caused by uncontrollable negative events. Prior to the present invention, it is not known to the skilled in the art whether GluR2-CT polypeptide can be used for treating depressive disorder.

In the present invention, the inventors have surprisingly found that GluR2-CT polypeptide is a promising medication candidate for treating depressive disorder. As proved by the example, in the classical anti-depressive disorder model of mouse forced swim test, GluR2-CT polypeptide of the present invention can rapidly and significantly decrease the immobility time of forced swimming mice and can maintain a good antidepressant effect within 24 hours. The experimental data have shown that compared with the positive antidepressant HNK (ketamine metabolite), the compound reported in the present invention has a clearer antidepressant effect, which proves that GluR2-CT polypeptide is expected to be developed into a new medication with rapid onset of action for treating various types of depressive disorder, especially largely decreasing the risk of suicide in patients with major depressive disorder due to its rapid onset of action.

In the present invention, “GluR2-C terminal polypeptide” or “GluR2-CT polypeptide” means a polypeptide derived from the C terminal of a GluR2 polypeptide capable of inhibiting NMDA-mediated excessive endocytosis of AMPAR or regulating neuronal apoptosis. The GluR2-CT polypeptide may be identical to the C-terminal of the receptor GluR2 polypeptide or may contain the substitution of one or more amino acids as compared thereto but retain its biological function. For example, GluR2-CT polypeptide comprises the amino acid sequence of YKEGYNVYG (SEQ ID NO: 1). However, GluR2-CT polypeptide may also comprise other polypeptide sequences in the form of fusion proteins, such as vector polypeptide sequences facilitating in penetrating cell membrane. The vector polypeptide can be for example a Tat polypeptide comprising the amino acid sequence of YGRKKRRQRRR (SEQ ID NO: 2). In some embodiments, the polypeptide is a separated polypeptide. In some embodiments, the polypeptide is an artificially synthetic polypeptide. In some embodiments, the polypeptide is a recombinant polypeptide.

Therefore, in a first aspect the present invention, provided is a method for treating depressive disorder in a subject, comprising administering to the subject a therapeutically effective amount of GluR2-CT polypeptide. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of the Tat carrier polypeptide at the N terminal. In some specific embodiments, the GluR2-CT polypeptide comprises the amino acid sequence of YGRKKRRQRRRYKEGYNVYG (SEQ ID NO: 3).

As used herein, the term “subject” refers to a mammal, especially a primate, in particular a human.

Another important finding of the present invention is that the GluR2-CT polypeptide acts rapidly in a depressive disorder animal model and is therefore particularly suitable for treating major depressive disorder as compared to conventional antidepressants. Therefore, in some embodiments of the method of the present invention, the depressive disorder is major depressive disorder.

The “depressive disorder” described herein can be clinically diagnosed and graded by a depression symptom rating scale. Appropriate scales include, but are not limited to Hamilton Depression Scale (HAMD), Beck Depression Inventory (BDI), and Self-rating Depressive disorder Scale (SDS).

For example, in HAMD scale, a total score less than 8 is considered normal, a total score of 8-20 indicates likely depressive disorder, whereas a total score of 20-35 is diagnosed as having depressive disorder, and a score of greater than 35 is diagnosed as having major depressive disorder. The GluR2-CT polypeptide of the invention is particularly suitable for patients with major depressive disorder who get a total score of greater than 35 in HAMD.

In BDI, a total score of 10-15 indicates likely depressive disorder; a total score of greater than 15 indicates depressive disorder, and a score of greater than 25 indicates major depressive disorder. The GluR2-CT polypeptide of the invention is particularly suitable for patients with major depressive disorder who get a total score of greater than 25 in BDI.

In SDS, the demarcation value of SDS standard score is 53, wherein a score of 53-62 indicates mild depressive disorder, a score of 63-72 indicates moderate depressive disorder, and a score of greater than 73 indicates major depressive disorder. The GluR2-CT polypeptide of the invention is particularly suitable for patients with major depressive disorder who get a standard score of greater than 73 in SDS.

Patients with major depressive disorder often have severe suicidality, resulting in serious social problems. Suicidality is, for example, that the subject has prepared for a suicidal action, or that the subject has committed a suicidal action but has been stopped. For such patients, it often takes several weeks for conventional antidepressants to act, which cannot effectively prevent suicidal behavior in patients. However, GluR2-CT polypeptide of the invention can rapidly act (for example, within 1 hour), and the effect lasts for a long time (for example, the effect of one dose can last for at least about 24 hours), which can reduce the suicidality in patients with major depressive disorder in time and prevent the occurrence of suicidal behavior.

Thus, in some embodiments of the present invention, the depressive disorder is major depressive disorder with suicidality. In some embodiments, the subject is administered to a therapeutically effective amount of GluR2-CT polypeptide when the subject indicates suicidality.

In another aspect, the present invention provides a method for decreasing the risk of suicide in a subject suffering from depressive disorder, comprising administering to the subject a therapeutically effective amount of GluR2-CT polypeptide. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of a Tat carrier polypeptide at the N terminal. In some specific embodiments, the GluR2-CT polypeptide comprises the amino acid sequence of YGRKKRRQRRRYKEGYNVYG (SEQ ID NO: 3).

In some embodiments, the depressive disorder is major depressive disorder.

In some embodiments, the depressive disorder is major depressive disorder with suicidality.

In some embodiments, the subject is administered with a therapeutically effective amount of GluR2-CT polypeptide when showing suicidality.

In another aspect, the present invention provides the use of GluR2-CT polypeptide in the preparation of a medication for treating depressive disorder in a subject. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of a Tat carrier polypeptide at the N terminal. In some specific embodiments, the GluR2-CT polypeptide comprises the amino acid sequence of YGRKKRRQRRRYKEGYNVYG (SEQ ID NO: 3).

In some embodiments, the depressive disorder is major depressive disorder.

In some embodiments, the depressive disorder is major depressive disorder with suicidality.

In some embodiments, the medication is used to be administered to a subject when the subject shows suicidality.

In another aspect, the present invention provides the use of GluR2-CT polypeptide in the preparation of a medication for decreasing the risk of suicide in a subject suffering from depressive disorder. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of a Tat carrier polypeptide at the N terminal. In some specific embodiments, the GluR2-CT polypeptide comprises the amino acid sequence of YGRKKRRQRRRYKEGYNVYG (SEQ ID NO: 3).

In some embodiments, the depressive disorder is major depressive disorder.

In some embodiments, the depressive disorder is major depressive disorder with suicidality.

In some embodiments, the medicine is used to be administered to a subject when the subject shows suicidality.

In another aspect, the present invention provides a pharmaceutical composition comprising a GluR2-CT polypeptide and a pharmaceutically acceptable carrier for treating depressive disorder in a subject. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of a Tat carrier polypeptide at the N terminal. In some specific embodiments, the GluR2-CT polypeptide comprises the amino acid sequence of YGRKKRRQRRRYKEGYNVYG (SEQ ID NO: 3).

In some embodiments, the depressive disorder is major depressive disorder.

In some embodiments, the depressive disorder is major depressive disorder with suicidality.

In some embodiments, the pharmaceutical composition is used to be administered to a subject when the subject shows suicidality.

In another aspect, the present invention provides a pharmaceutical composition comprising a GluR2-CT polypeptide and a pharmaceutically acceptable carrier for decreasing the risk of suicide in a subject suffering from depressive disorder. In some embodiments, the GluR2-CT polypeptide comprises the amino acid sequence of YKEGYNVYG (SEQ ID NO: 1). In some embodiments, the GluR2-CT polypeptide is fused with a carrier polypeptide sequence at the N terminal. In some embodiments, the GluR2-CT polypeptide is fused with the amino acid sequence of YGRKKRRQRRR (SEQ ID NO: 2) of a Tat carrier polypeptide at the N terminal. In some specific embodiments, the GluR2-CT polypeptide comprises the amino acid sequence of YGRKKRRQRRRYKEGYNVYG (SEQ ID NO: 3).

In some embodiments, the depressive disorder is major depressive disorder.

In some embodiments, the depressive disorder is major depressive disorder with suicidality.

In some embodiments, the medication is used to be administered to a subject when the subject shows suicidality.

As described herein, a “therapeutically effective amount” of GluR2-CT polypeptide preferably results in a decrease in the severity of symptoms, an increase in the frequency and duration of the asymptomatic period of the disorder, or the prevention of injury or disability as a result of suffering from the disorder. For example, administration of a therapeutically effective amount of GluR2-CT polypeptide may lower the score rated in a depressive disorder scale for the subject. For example, administration of a therapeutically effective amount of GluR2-CT polypeptide may result in a decrease of about 5, about 10, about 15, about 20 or more in HAMD total score, or a decrease of about 5, about 10, about 15, about 20 or more in BDI total score, or a decrease of about 5, about 10, about 15, about 20 or more in SDS standard score. Preferably, administration of a therapeutically effective amount of GluR2-CT polypeptide may result in remission from major depressive disorder to mild depressive disorder or no depressive symptoms in a subject. Alternatively, administration of a therapeutically effective amount of GluR2-CT polypeptide may result in a decreased risk of suicide in a subject. The skilled in the art may determine a therapeutically effective amount based on factors such as age and severity of symptoms (e.g., scoring high or low) of the subject, and the selected specific composition or route of administration.

The GluR2-CT polypeptide or pharmaceutical composition of the present invention can be administered by one or more routes of administration using one or more methods known in the art. It will be understood by the skilled in the art that the route and/or manner of administration may vary depending on the desired result. Preferred routes of administration of GluR2-CT polypeptide of the present invention include intravenous, intramuscular, intradermal, intraperitoneal, subcutaneous, spinal, or other parenteral administration, such as injection or infusion. Preferably, the GluR2-CT polypeptide of the present invention is administered by injection.

For the administration of the GluR2-CT polypeptide of the present invention, the dose range may be from about 0.0001 to 100 mg/kg body weight, and more typically from 0.01 to 20 mg/kg body weight of a subject. For example, the dose may be 1 mg/kg body weight, 3 mg/kg body weight, 5 mg/kg body weight, 10 mg/kg body weight or 20 mg/kg body weight, or in the range of 1-20 mg/kg body weight.

For the administration of the GluR2-CT polypeptide of the present invention, exemplary treatment scheme may be administration once a day, once every two days, once every three days, once every four days, once every five days, once every six days, once every week, once every two weeks, once every three weeks, once every month, once every three months, once every 3-6 months, or with shorter intervals for initial administrations (e.g. once every day to once every three weeks) and longer intervals for later administrations (e.g. once every month to once every 3-6 months). The specific administration scheme can be determined by doctors based on the specific symptoms of patients. For example, as described above, a patient can be administered with the GluR2-CT polypeptide of the present invention when developing major depressive disorder, such as suicidality, thereby rapidly decreasing the risk of suicide in the patient. At the same time, the GluR2-CT polypeptide of the invention has a long duration of efficacy, allowing for a significantly reduced frequency of administration.

As used herein, “pharmaceutically acceptable carriers” include any and all physiologically compatible solvents, dispersion medium, coatings, antibacterial and antifungal agents, isotonic agents and absorption retarders, etc. Preferably, the carrier is suitable for intravenous, intramuscular, intradermal, intraperitoneal, subcutaneous, spinal, or other parenteral administration (e.g., by injection or infusion). Depending on the route of administration, an active compound, i.e., GluR2-CT polypeptide, can be encapsulated in a material to protect the compound from acids and other natural conditions that may inactivate the compound.

### EXAMPLES

The following examples are used to further illustrate the present invention, but the scope of the present invention is not limited to these examples.

At present, methods for establishing animal model of depressive disorder are mainly divided into three categories: behavioral despair, learned helplessness and chronic mild unpredictable stress.

Among the behavioral despair models, forced swim test of mice/rats is the most common animal model of depressive disorder to evaluate the effect of antidepressants. In this model, animals are forced to swim in water in a limited space. After struggling for many times but failing to escape, the animals may stop swimming and begin floating without struggling to escape anymore. This immobility is considered as the manifestation of behavioral despair and depression. This model has good predictive validity for antidepressants.

### 1. Experimental Purpose

By using ketamine metabolite HNK as a positive control medication, the effect of compound PMS-001 on depression-like behavior of C57 mice was investigated through forced swim test 1 hour and 24 hours after single medicinal intervention. Compound PMS-001 (also known as Tat-GluR2-3Y) is a polypeptide compound comprising the amino acid sequence of Tyr-Gly-Arg-Lys-Lys-Arg-Arg-Gln-Arg-Arg-Arg-Tyr-Lys-Glu-Gly-Tyr-Asn-Val-Tyr-Gly (YGRKKRRQRRRYKEGYNVYG; (SEQ ID NO: 3). Wherein the amino acid sequence YGRKKRRQRRR (SEQ ID NO: 2), also known as Tat peptide, is a carrier peptide, and can assist the polypeptide in penetration of the blood-brain barrier. The amino acid sequence YKEGYNVYG (SEQ ID NO: 1), also known as GluR2-3Y, is derived from the C-terminal sequence of GluR2, and has been proved to inhibit NMDA-mediated excessive endocytosis of AMPAR, thus regulating neural response (see EP1,687,427B1). However, it is unknown in the art whether a C-terminal polypeptide of GluR2 such as YKEGYNVYG (SEQ ID NO: 1) can be used in the treatment of depressive disorder, specifically major depressive disorder.

### 2. Protocol

### 1) Animals

The experimental animals were 6-week-old male C57 mice obtained from Shanghai SLAC Laboratory Animal Co., Ltd, with body weight of 20.45±9 g. They were transported to the Animal Breeding Center of Shanghai Institute of Materia Medica, Chinese Academy of Sciences (Animal production license: SCXK9 [Shanghai] 2004-0002, use license: SYXK [Shanghai] 2003-0029), and allowed to acclimatize in animal facilities for more than 3 days, with 6 individuals per cage. Mice were housed at room temperature (23±0.2° C.), with a 12 h/12 h light/dark cycle. Before the behavioral test, animals were moved to the behavior testing room 2 hours in advance for acclimation to relieve tension.

### 2) Medication and Reagents

Reagents under test in this study include HNK, PMS 001, and 0.9% NaCl solution (blank control) were. The specific information of each tested reagent is shown in Table 1 below:

TABLE 1
Tested Medications

| | | | Total weight | | Molecular | | |
|---|---|---|---|---|---|---|---|
| Sample | Batch No. | Supplier | of sample | | weight | Purity | Storage condition |
| NaCl | 101005408 | Sigma | 5 | KG | 58.44 | ≥99.5% | Room temperature |
| HNK | 20161011 | | 5.73 | | 276.73 | Null | Room temperature |
| PMS 001 | Null | | 20 | mg | | Null | Room temperature |

### 3) Instruments

1 mL sterile disposable syringe with needle (available from Zhejiang Kangdelai Medical Devices Co., Ltd.); transparent tanks (self-made, 30 cm in height, 20 cm in diameter with 15 cm of water depth), device for forced swim of mice; HD camera; and Jiliang behavior analysis system (Shanghai Jiliang Software Technology Co., Ltd.).

### 4) Grouping

| | | | Dose of | Volume of |
|---|---|---|---|---|
| | Animal | Route of | injection | injection |
| Group | numbers | Administration | (mg/kg) | (ml/kg) |
| Vehicle | 10 | Intraperitoneal | 0.9% NaCl | 7.5 |
| control group | | injection | | |
| HNK | 10 | Intraperitoneal | 10 | 7.5 |
| | | injection | | |
| PMS 001 | 10 | Intraperitoneal | 10 | 7.5 |
| | | injection | | |

### 5) Behavioral Test

### (1) Experimental Procedures

i) Formulation of medications to be tested (fresh formulation before each experiment): HNK and PMS-001 powders of a certain amount were taken and 4 mg of each was precisely weighed to dilute to volume in 3 mL of 0.9% NaCl, respectively, mixed well, and formulated as 1 mg/mL solution for later use.
ii) Animals were randomly divided into 6 groups, 10 animals per group. The routes of administration for animals of each group were shown in Table 2.
iii) Before test, mice were placed in the testing room for an acclimation period of more than 2 hours.
iv) Before acclimation, mice were weighed and marked on the tails. One hour before the forced swim test, each animal was given medicinal intervention, as shown in Table 2.

### (2) Forced Swim Test (FST)

24 hours before administration, each mouse was placed into a cylinder tank for acclimation in the water environment for 10 min. Animals were administrated intraperitoneally 24 hours before the behavior test, and once 1 hour before the test on the day of test, respectively. Each mouse was separately placed into a cylindrical glass tank (30 cm in height and 20 cm in diameter) filled with water of 15 cm depth such that the animals would neither escape from the glass tank nor touch the bottom of the tank either with their feet or tails, and the water temperature was 23° C.-25° C. Mice were videotaped for 6 minutes since being placed into water, and the immobility time for the last 4 minutes was calculated due to the fact that most animals were very active in the first two minutes (the criteria for immobility: mice stop struggling, keep immobility and make small movements to balance their bodies or float in water). Mice in each group were tested in parallel.

### 6) Data Processing and Statistical Methods

All data analyses were performed with spss 22 data processing software. With one-way analysis of variance, multiple comparisons were conducted by post-hoc LSD method to verify the results. Data were expressed as Mean±SEM. *: p<0.05; **: p<0.01; ***: p<0.001.

### 3. Experimental Results

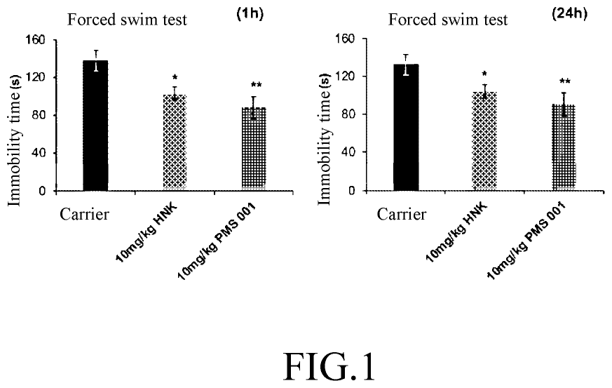

Due to the slow onset of action of conventional antidepressants, administration of a single dose cannot produce an antidepressant effect. In this experiment, ketamine metabolite HNK was used as positive medication because it exerts a rapid onset of action within 1 hour after administration and is efficacious for 24 hours as shown in previous studies. The results of this experiment showed that (FIG. 1), 1 hour after administration, the tested compound HNK could significantly decrease the immobility time of forced swimming mice, whereas PMS-001 could very significantly decrease that of forced swimming mice. Behavioral test was conducted 24 hours after administration, and it was found that HNK could significantly decrease the immobility time of the forced swimming mice, whereas PMS-001 could very significantly decrease that of the forced swimming mice. The results basically proved that HNK and PMS-001 had antidepressant effects and were efficacious for up to 24 hours.

### 4. Conclusions

The depressive disorder model-forced swim test has shown that PMS-001 manifests a better rapid and lasting antidepressant effect (the antidepressant effect occurs 1 hour after administration and lasts for 24 hours) compared to the positive control ketamine metabolite HNK at the same dose.

The animal model experiment has shown that PMS-001 can act rapidly, thereby decreasing the onset time as compared to that of conventional medications on the market (about 4 weeks), and reducing the treatment period for patients, thus greatly decreasing the risk of suicide in patients suffering from major depressive disorder as well as family and social problems caused by depressive disorder.

1-9. (canceled)

10. A method for treating depressive disorder or reducing the risk of suicide in a subject suffering from depressive disorder, the method comprising administering to the subject an effective amount of a composition comprising a GluR2-CT polypeptide comprising the amino acid sequence YKEGYNVYG (SEQ ID NO: 1).

11. The method of claim 10, wherein the GluR2-CT polypeptide further comprises the amino acid sequence of YGRKKRRQRRR (SEQ ID NO: 2) fused to its N-terminus.

12. The method of claim 11, wherein the GluR2-CT polypeptide comprises the amino acid sequence YGRKKRRQRRRYKEGYNVYG (SEQ ID NO: 3).

13. The method of claim 1, wherein the depressive disorder is major depressive disorder.

14. The method of claim 1, wherein the subject has a Hamilton Depression Scale (HAMD) score of greater than 35, a Hamilton Depression Scale (HAMD), Beck Depression Inventory (BDI) score of greater than 25, or a Self-rating Depressive disorder Scale (SDS) standard score of greater than 73.

15. The method of claim 1, wherein the depressive disorder is major depressive disorder with suicidality.

16. The method of claim 1, wherein the composition is administered to the subject when the subject shows suicidality.

17. The method of claim 1, wherein the composition comprises the GluR2-CT polypeptide at a dose of 1-20 mg/kg body weight.

18. The method of claim 1, wherein the composition is administrated by injection.
